(19) United States
(12) Patent Application Publication
(10) Pub. No.: US 2021/0186062 A1
(43) Pub. Date: Jun. 24, 2021

(54) METHODS AND SYSTEMS FOR STERILIZING PLANT MATERIALS AND PRODUCTS

(71) Applicant: Maui Wellness Group, LLC, Los Angeles, CA (US)

(72) Inventor: Christopher A. Cole, Kula, HI (US)

(21) Appl. No.: 16/721,749

(22) Filed: Dec. 19, 2019

(51) Int. Cl. A23L 3/015; A23B 7/005; A23L 3/16; A61L 2/00

(52) U.S. Cl. CPC A23L 3/0155; A23B 7/0053; A61L 2202/14; A61L 2/0023; A23V 2002/00; A23L 3/165

(57) ABSTRACT

A variety of plant products that are intended for consumption can become contaminated with microbial growth. The levels of contamination must be reduced or eliminated in order for the product to pass microbial growth testing standards. The contaminated plant product can be sterilized using high pressure processing to treat the product with a pressurized gas at a temperature for a desired period of time to reduce the level of the microbial contaminants present in the plant product to a predetermined acceptable level. After treatment, the sterilized plant product can then pass microbial testing standards and be fit for consumption.

### TECHNICAL FIELD

The field relates to methods and systems for sterilizing herbaceous and woody plant materials and products intended for human use and consumption, including flowers, fruits, leaves, seeds, barks, and roots using pressure processing, and particularly processing with a highly pressurized gas. The plant products can become contaminant free and safe for human use and consumption, and in compliance with applicable standards.

### BACKGROUND OF THE INVENTION

Examples of plant materials and products intended for human consumption include flowers, fruits, leaves, seeds, barks and roots (collectively referred to herein as “plant products”). As used herein, the term “human consumption” means ingestion by any means, for example, by swallowing, inhalation or by absorption through the skin. Environmental molds and yeasts, as well as aerobic bacteria, are present in virtually every plant product production environment. Accordingly, plant products can become contaminated with microbes during growth and/or during processing. For example, microbial growth is very common in climates that have conditions conducive to microbial growth, such as climates having high humidity and warm temperatures. A person who consumes a contaminated plant product can become ill from microbe contamination. To prevent such ill effects, those products undergo microbial testing to ensure the products are not contaminated. Common microbes that are tested include, but are not limited to Aspergillus, Salmonella, E. coli, aerobic bacteria, enterobacteria, coliforms, yeasts, and molds.

The most common microbial testing procedure is to determine the number of colony-forming units (CFU) in a sample. A CFU is a unit used to estimate the number of viable (i.e., cells that multiply via binary fission) bacterial or fungal cells in a sample. The sample to be tested is prepared and possibly diluted and generally streaked onto an agar growth medium in a Petri dish for example. The agar plate is then allowed to incubate for a specific period of time. After the incubation period has elapsed, the number of colonies that have formed can be counted in a variety of ways. The results are then reported in units of CFU/mL for liquid samples or CFU/g for solid samples. Different standards exist which determine whether a sample fails or passes the microbial testing procedure. The results must be less than the predetermined value of CFU/mL or CFU/g in order for a sample to pass. For example, according to one standard, yeast and mold must be present in the sample less than 100 CFU/g in order to pass microbial testing. It is possible for a sample to pass with the presence of some microbes, but not others.

Plant products subject to microbial testing can be sterilized. As used herein, the term “sterilized” and all grammatical variations thereof means the reduction or elimination of microbes such that the product will pass microbial testing. Some sterilization procedures—for example, the use of heat, oxidizing agents such as ozone, gamma irradiation, or radio waves—will sterilize the products, but at the expense of loss of product quality and degradation of favorable components such as volatile organic compounds in the product. Other sterilization processes have been used for liquids without a solution for solids. Thus, there is a need and ongoing industry-wide concern for sterilization procedures that can be used for solid plant products that will not degrade the valuable therapeutic, organoleptic or nutritional properties of the plant products.

### SUMMARY OF THE INVENTION

A novel system and method uses high pressure processing (HPP) in combination with food grade gas and temperature control to sterilize plant products such that the plant products pass applicable microbial testing standards and are made safe for consumption. The plant product's valuable properties are also maintained.

According to certain embodiments, a method of sterilizing a plant product comprises: testing the plant product to determine the presence of microbial contaminants; and treating the plant product by subjecting the plant product to a pressurized gas at a temperature for a desired period of time appropriate to reduce the presence of the detected microbial contaminants to acceptable levels.

According to other embodiments, a system for sterilizing a plant product comprises: a pressure vessel, wherein the pressure vessel holds a gas at a desired pressure; a temperature controller for adjusting the temperature inside the pressure vessel; the plant product, wherein the plant product is sterilized by contacting the plant product with the pressurized gas within the pressure vessel at a temperature for a desired period of time appropriate to reduce the presence of the detected microbial contaminants to acceptable levels.

### DETAILED DESCRIPTION OF THE INVENTION

It is to be understood that any discussion of any of the components disclosed herein is meant to apply to the method and system embodiments without the need to repeat information throughout. By way of example, any discussion related to the plant products and the treatment thereof is meant to apply to the method and system embodiments.

The methods include sterilizing a plant product. The plant product can be intended for human or animal consumption. According to certain embodiments, the sterilization process allows a sample to pass microbial testing according to a variety of testing standards. The sterilization process can be high-pressure processing (HPP). According to certain embodiments, the plant product is a flower. According to certain embodiments, the flower is dried prior to treating the flower. As used herein, the term “dry” and all grammatical variations thereof means the flower has a moisture content less than 20%, preferably less than 15% by weight of the flower. The flower can be any flower that is intended for consumption. By way of a non-limiting example, the flower can be a Cannabis flower (also called a bud) or a hops flower (also called seed cones or strobiles). Cannabis flowers can be used to provide many therapeutic benefits to users, while hops flowers can be used in brewing of beer. Cannabis refers to a group of three plants, Cannabis sativa, Cannabis indica, and Cannabis ruderalis. There can be over 100 different phytochemical components in Cannabis. The exact components as well as the levels of each component can differ between cultivars. For example, one cultivar can have a higher level of cannabidiol (CBD) and lower levels of tetrahydrocannabinol (THC); whereas a different cultivar may have higher levels of THC. Hemp, for example, which is a strain of C. sativa, has virtually no THC 0.3%).

According to certain embodiments, the plant product is a fruit. The fruit may be in a solid form (i.e., not a liquid form). The fruit can also be dried. The fruit can be any fruit that requires sterilization to be fit for consumption. Some fruits have a protective covering that surrounds the flesh to be consumed. Coconuts and bananas, for example, have a covering that surrounds the flesh which is to be consumed. These types of fruits may not need sterilization because the flesh or meat interior may not be exposed to microbes. According to certain embodiments, the fruit either has a protective covering that is intended to be consumed or does not have a protective covering. Non-limiting examples of fruits that have a covering that is intended to be consumed include cherries, apples, peaches, plums, pluots, cranberries, figs, apricots, grapes, blueberries, pears, and nectarines. Non-limiting examples of fruits that do not have a protective covering include aggregate fruits, such as strawberries, blackberries, and raspberries. In other embodiments, the plant product is edible herbs, barks, leaves, seeds, nuts, vegetables, mushrooms, and other edible plant products.

The methods can further include providing the plant product. The plant product can be purchased, transported, or locally grown and harvested. The plant product is tested to determine the presence of microbial contaminants. The plant product is then treated by subjecting the plant product to a pressurized gas at a temperature for a desired period of time appropriate to reduce the presence of the detected microbial contaminants to acceptable levels.

The methods can further include placing the plant product into a pressure vessel prior to treating. The pressure vessel can include one or more gas inlets and a control switch for causing and maintaining a desired pressure within the pressure vessel. The methods can further include pressurizing and maintaining the pressure in the pressure vessel. This can be accomplished, for example, by opening a valve to allow the gas to flow into the pressure vessel and selecting the desired pressure via the control switch. According to certain embodiments, the gas is an inert gas. The inert gas can be a food grade gas. An inert gas is a gas that has very low to no chemical reactivity with other compounds. By way of example, the inert gas can be carbon dioxide or nitrogen. The specific inert gas used can be selected based on the type of microbial contaminants found in the plant product. For example, nitrogen may be used when the detected microbial contaminants in the pre-treatment plant product are mainly aerobic bacteria, which require an oxygenated environment to remain viable.

The desired pressure can be selected such that the plant product is sterilized to reduce the level of microbial contaminants to acceptable levels. The desired pressure may be related to one or more of the other treatment conditions. By way of example, the pressure may be inversely related to the period of time. Accordingly, the desired pressure may be reduced with an increased period of time or increased with a reduced period of time. According to certain embodiments, the desired pressure is selected such that the structural integrity and the favorable components of the plant product are maintained. According to these embodiments, the desired pressure may need to be reduced in order to maintain the structural integrity and favorable components. As discussed above, if the desired pressure is reduced, then the period of time may need to be increased in order to sterilize the plant product. According to certain embodiments, the desired pressure is in a range of 1,200 pound-force per square inch (psi) to 8,700 psi (8.27 megapascal (MPa) to 59.98 MPa). According to certain other embodiments, for plant products that may be damaged by higher pressures, the desired pressure is in a range from 1,200 psi to 3,000 psi (8.27 MPa to 20.68 MPa).

The plant product is treated at a temperature. The temperature in the pressure vessel can be controlled by a variety of mechanisms (e.g., a water cooling jacket, a recirculating bath, and a temperature controller) those of ordinary skill in the art will appreciate. The methods can further include selecting and maintaining the desired temperature in the pressure vessel via the temperature controller. The temperature can be selected such that favorable components of the plant product are not degraded. By way of example, some of the favorable components of a Cannabis flower, such as the organic compounds called terpenoids, can be degraded at temperatures exceeding 80° F. (27° C.). The temperature can also be selected such that microbes present on the plant product are killed in order to sterilize the plant product. For example, if the temperature is too low, then a sufficient number of the microbes may survive. According to certain embodiments, the temperature is in the range of 75° F. to 122° F. (24° C. to 50° C.). The temperature can also be in the range of 77° F. to 80° F. (25° C. to 27° C.).

The plant product is treated for a desired period of time. According to certain embodiments, the desired period of time is selected such that the plant product is sterilized based on the microbial contaminants found in the plant product. The period of time for treating the plant product may be directly related to the level of microbial contamination. For example, at higher levels of microbial contamination or for microbes that are more robust, the period of time may need to be lengthened in order to sterilize the plant product. The period of time can also be selected such that the structural integrity and the favorable components of the plant product are maintained. The structural integrity and the favorable components may become degraded with longer periods of time. As discussed above, the time can be inversely related to the pressure. Accordingly, depending on the specific pressure, the period of time can be lengthened or shortened. According to certain embodiments, the desired period of time is in the range of 3 minutes (min) to 360 min. The period of time can also be in the range of 5 min to 60 min. At the end of the period of time, the pressure and temperature are returned to ambient conditions and the plant product can be removed from the pressure vessel.

Each of the treatment conditions (i.e., pressure, temperature, time period, and type of gas used) can vary and be selected based on the characteristics of the plant product being treated and the types of microbial contaminants detected in the pre-treatment plant product and to achieve sterilization, structural integrity, and retention of favorable components of the plant product. By way of example, the treatment conditions can be adjusted for a Cannabis flower depending on the levels of the favorable components in a particular cultivar of Cannabis. A cultivar having higher levels of terpenoids may require different treatment conditions compared to a cultivar having very low levels of terpenoids.

The following are examples of varying treatment conditions based in part on the type of plant product, the type of microbe detected, and/or the level of microbial contamination. One of ordinary skill in the art will be able to select the appropriate treatment conditions based on the teachings set forth herein. A sample of Cannabis flowers is analyzed by culture-based microbial testing and found to contain a total yeast and mold count exceeding the applicable regulatory standard by a factor of ten. The corresponding batch of Cannabis flowers is subjected to treatment with food grade carbon dioxide (CO₂) pressurized to 1,200 psi (8.27 MPa) at 80.6° F. (27° C.) for 60 min. The food grade CO₂ and relatively low temperature and pressure can be selected because common environmental yeasts and molds are generally labile at low pressures and temperatures; and the low pressures and temperatures may be preferable when dealing with fragile matrices like Cannabis flowers. A sample from the treated batch of Cannabis flowers is then analyzed by the same culture-based microbial testing method used to analyze the untreated Cannabis flowers. As a result of treatment, the total yeast and mold count on the treated sample will be reduced by a factor of greater than 10 compared to the untreated sample. Accordingly, the corresponding batch of treated Cannabis flowers will be sterilized and meet the applicable regulatory requirement for sale as produce intended for human consumption.

A sample of hops is analyzed by culture-based microbial testing and found to contain an aerobic bacteria count exceeding the applicable regulatory standard by a factor of 100. The corresponding batch of hops is subjected to treatment with food grade nitrogen (N₂) gas pressurized to 1,200 psi (8.27 MPa) at 77° F. (25° C.) for 30 min. The food grade N₂ can be selected because aerobic bacteria require an oxygenated environment to remain active. As with yeasts and molds, a lower pressure can be selected because aerobic bacteria are generally labile at relatively low pressures. The temperature and time of 25° C. and 30 min can be selected because temperature and time do not appear to be as important as the pressure and type of gas selected when aerobic bacteria are the treatment targets. A sample from the treated batch of hops is then analyzed using the same culture-based microbial testing method used to analyze the untreated hops. As a result of treatment, the aerobic bacteria count on the treated sample will be reduced by a factor of greater than 100 compared to the untreated sample. Accordingly, the corresponding batch of treated hops will be sterilized and meet the applicable regulatory requirement for sale as produce intended for human consumption.

A sample of flax seed is analyzed by culture-based microbial testing and found to contain E. coli. The corresponding batch of flax seed is subjected to treatment with food grade CO₂ pressurized to 8,700 psi (59.98 MPa) at 122° F. (50° C.) for 60 min. This much higher pressure and temperature in the E. coli example, as compared with the two examples above, can be selected because: E. coli is known to be highly pathogenic in humans and is bile-tolerant (i.e., it can be swallowed, survive the acidic conditions of the stomach, and go on to infect human organisms); and generally in most jurisdictions, any amount of E. coli on any type of produce will make that produce unsalable. Therefore, treatment should be targeted to not just reduce the number of E. coli CFUs in the plant product—it should completely eliminate any trace of the organism. Moreover, the harder seed matrix of the flax seed allows the seed to withstand higher pressures without any negative structural impact. A sample from the treated batch of flax seed is then analyzed using the same culture-based microbial testing method used to analyze the untreated flax seed. As a result of treatment, no E. coli will be present on the treated sample. Accordingly, the corresponding batch of treated flax seed will be sterilized and meet the applicable regulatory requirement for sale as produce intended for human consumption.

A sample of macadamia nuts is analyzed by culture-based microbial testing and found to contain Salmonella. The corresponding batch of macadamia nuts is subjected to treatment with food grade CO₂ pressurized to 8,700 psi (59.98 MPa) at 96.8° F. (36° C.) for 30 min. The same higher pressure as with E. coli can be selected for Salmonella because generally all traces of Salmonella should be completely eliminated. The shortened length of time can be selected because Salmonella is generally less pressure-tolerant and thermo-tolerant than E. coli and; therefore, does not require as much time for complete elimination if pressure and temperature are constant. Again, the solid nut matrix allows for higher pressures with no negative structural impact. A sample from the treated batch of macadamia nuts is then analyzed using the same culture-based microbial testing method used to analyze the untreated macadamia nuts. As a result of treatment, no Salmonella will be present on the treated sample. Accordingly, the corresponding batch of treated macadamia nuts will be sterilized and meet the applicable regulatory requirement for sale as produce intended for human consumption.

The methods can further include further testing of the plant product after the treatment has been performed. Microbial testing can be performed to determine if the plant product is sterilized. In the event the plant product is not sterilized, the plant product may be treated again with the same or different treatment conditions. For example, the pressure and/or period of time may need to be increased. The sterilized plant product can be packaged to prevent new microbial contamination. The sterilized plant product can be stored, further processed, or transported.

### Examples

To facilitate a better understanding of the present invention, the following examples of certain aspects of various embodiments are given. The following examples are not the only examples that could be given according to the present invention and are not intended to limit the scope of the invention.

A sample Cannabis sativa L flower of the variety ‘Kolossus’ having 6.63 milligrams per dried gram (mg/g) tetrahydrocannabinol, 189.30 mg/g tetrahydrocannabinolic acid, 0.38 mg/g cannabidiolic acid, 1.60 mg/g cannabinol, and 23.56 mg/g cannabigerol underwent microbial testing wherein microbial contamination was found to be present. The batch of flowers was then treated at a pressure of 900 psi (6.21 MPa) at 77° F. (25° C.) for 60 minutes. Another sample was tested after treatment for microbial growth. The results of the pre- and post-treatment microbial testing are shown in Table 1. As can be seen, pre-treatment testing showed high levels of yeast, mold, and aerobic bacteria wherein the sample failed the testing standards. However, after treatment, the number of microbial contaminants was significantly reduced, which allowed the sample to pass the testing standards. This indicates that the methods can be used to reduce microbial contamination to acceptable levels, thereby enabling the flower to meet the applicable regulatory requirement for sale as produce intended for human consumption.

TABLE 1

| Microbial | Pre-Treatment | Post-Treatment |
|---|---|---|
| Contaminate | (CFU/g) | (CFU/g) |
| Yeast | 39,000 | 300 |
| Mold | 39,000 | 300 |
| Aerobic Bacteria | 21,000 | 1,000 |

A sample of Cannabis sativa L flower of the variety Blood Oranges having 18.3 mg/g tetrahydrocannabinol, 220 mg/g tetrahydrocannabinolic acid, 9.5 mg/g cannabidiolic acid and 11.6 mg/g tetrahydrocannabivarinic acid underwent microbial testing wherein microbial contamination was found to be present. The batch of flowers was then treated at a pressure of 1,900 psi (13.1 MPa) at 122° F. (50° C.) for 360 minutes. Another sample was tested following treatment using the methods described herein. The results of the pre- and post-treatment microbial testing are shown in Table 2. As can be seen, pre-treatment testing showed the presence of Aspergillus fumigatus, a species of fungus known to be pathogenic in humans and highly thermotolerant, along with high levels of yeast, mold, aerobic bacteria, coliforms, and enterobacteria. However, after treatment, no Aspergillus fumigatus was detected, while the numbers of colony forming units of yeast, mold, aerobic bacteria, coliforms, and enterobacteria were reduced to below the limits of quantitation, as shown in Table 2. This indicates that the methods can be used to completely inactivate the thermotolerant pathogenic fungal species Aspergillus fumigatus, while also significantly reducing the overall pre-treatment microbial load, thereby enabling the plant material to meet the applicable regulatory requirements for sale as produce intended for human consumption.

TABLE 2

| Microbial | Pre-Treatment | Post-Treatment |
|---|---|---|
| Contaminate | (CFU/g) | (CFU/g) |
| Aspergillus fumigatus | Detected | Not detected |
| Aerobic bacteria | 350,445 | <100 |
| Coliform | 3,409 | <100 |
| Enterobacteria | 38,833 | <100 |
| Yeast & Mold | 64,406 | <100 |

A sample of Cannabis sativa L flower of the variety Chocolope having 29 mg/g tetrahydrocannabinol, 210 mg/g tetrahydrocannabinolic acid, 4.8 mg/g cannabidiolic acid, 12 mg/g cannabigerol, and 10.5 mg/g tetrahydrocannabivarinic acid underwent microbial testing wherein contamination was found to be present. The batch of flowers was then treated at 1,900 psi (12.4 MPa) at 122° F. (50° C.) for 240 minutes. Another sample was tested for the presence of microbial contaminants following treatment. The results of the pre- and post-treatment microbial testing are shown in Table 3. As can be seen, pre-treatment microbial testing showed the presence of coliforms in amounts exceeding the applicable regulatory requirements, as well as the presence of fungus of the genus Aspergillus. However, following treatment using the methods disclosed herein, the amounts of aerobic bacteria and coliforms were reduced to below the limit of quantitation, and no fungus of the genus Aspergillus was detected, thereby enabling the batch of flowers to meet the applicable regulatory requirements for sale as produce intended for human consumption.

TABLE 3

| Microbial | Pre-Treatment | Post-Treatment |
|---|---|---|
| Contaminate | (CFU/g) | (CFU/g) |
| Aspergillus fumigatus | Detected | Not detected |
| Aerobic bacteria | 70,330 | <100 |
| Enterobacteria | 3,323 | <100 |
| Yeast & Mold | 9,274 | 8,127 |

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention.

As used herein, the words “comprise,” “have,” “include,” and all grammatical variations thereof are each intended to have an open, non-limiting meaning that does not exclude additional elements or steps. While compositions, systems, and methods are described in terms of “comprising,” “containing,” or “including” various components or steps, the compositions, systems, and methods also can “consist essentially of” or “consist of” the various components and steps. It should also be understood that, as used herein, “first,” “second,” and “third,” are assigned arbitrarily and are merely intended to differentiate between two or more treatment conditions, etc., as the case may be, and does not indicate any sequence. Furthermore, it is to be understood that the mere use of the word “first” does not require that there be any “second,” and the mere use of the word “second” does not require that there be any “third,” etc.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

What is claimed is:

1. A method of sterilizing a plant product comprising:
determining microbial contaminants present in the plant product; and
treating the plant product by subjecting the plant product to a pressurized gas at a temperature for a desired period of time to reduce the level of the microbial contaminants to a predetermined acceptable level.

2. The method according to claim 1, wherein the plant product is a flower.

3. The method according to claim 2, wherein the flower is a Cannabis flower.

4. The method according to claim 1, wherein the plant product is a fruit, edible herbs, barks, leaves, seeds, nuts, vegetables, or mushrooms.

5. The method according to claim 1, wherein the gas is an inert gas selected from carbon dioxide or nitrogen.

6. The method according to claim 5, wherein the microbial contaminate is aerobic bacteria and wherein the gas is nitrogen.

7. The method according to claim 1, wherein the pressure, the temperature, and the desired period of time are selected such that structural integrity and favorable components of the plant product are maintained.

8. The method according to claim 1, wherein the pressure and the desired period of time are inversely related.

9. The method according to claim 1, wherein the pressure is in a range of 1,200 psi to 8,700 psi.

10. The method according to claim 1, wherein the temperature is in the range of 75° F. to 122° F.

11. The method according to claim 1, wherein the desired period of time is in the range of 3 minutes to 360 minutes.

12. The method according to claim 1, wherein the pressure, temperature, period of time, and type of gas used are selected based on the type of plant product being treated and the types of microbial contaminants detected in the plant product.

13. The method according to claim 1, wherein after treating the plant product, E. coli and Salmonella are not detected.

14. A system for sterilizing a plant product comprising:
a pressure vessel, wherein the pressure vessel holds a gas at a desired pressure;
a temperature controller for adjusting the temperature inside the pressure vessel;
the plant product, wherein the plant product is sterilized by contacting the plant product with the pressurized gas within the pressure vessel at a temperature for a desired period of time to reduce the level of the microbial contaminants present in the plant product to a predetermined acceptable level.

15. The system according to claim 14, wherein the pressure vessel comprises a gas inlet and a control switch for causing and holding a desired pressure within the pressure vessel.

16. The system according to claim 14, further comprising a water cooling jacket or a recirculating bath for adjusting the temperature inside the pressure vessel.

17. The system according to claim 14, wherein the plant product is a Cannabis flower.

18. The system according to claim 14, wherein the plant product is a fruit, edible herbs, barks, leaves, seeds, nuts, vegetables, or mushrooms.

19. The system according to claim 14, wherein the gas is an inert gas selected from carbon dioxide or nitrogen.

20. The system according to claim 14, wherein the pressure is in a range of 1,200 psi to 8,700 psi, the temperature is in the range of 75° F. to 122° F., and the desired period of time is in the range of 3 minutes to 360 minutes.
